(19) United States
(12) Patent Application Publication
(10) Pub. No.: US 2009/0232903 A1
(43) Pub. Date: Sep. 17, 2009

(54) BIOCIDE COMPOSITIONS

(76) Inventor: William D. Sanderson, Laguna Beach, CA (US)

Correspondence Address: MANELLI DENISON & SELTER, 2000 M STREET NW SUITE 700, WASHINGTON, DC, 20036-3307, US

(21) Appl. No.: 12/472,649

(22) Filed: May 27, 2009

Related U.S. Application Data
(63) Continuation-in-part of application No. 12/091,785, filed on Apr. 28, 2008.
(60) Provisional application No. 60/750,786, filed on Dec. 16, 2005.
(60) Provisional application No. 60/812,632, filed on Jun. 12, 2006.

(51) Int. Cl. A61K 33/42; A61K 33/40; A01N 59/08

(52) U.S. Cl. 424/601; 424/613; 424/616; 424/661

(57) ABSTRACT

Compositions and methods that yield chlorine dioxide upon mixing with an aqueous solution.

This application is a Continuation-in-Part of U.S. patent application Ser. No. 12/091,785, filed Dec. 15, 2006, publication No. 2008/0299161, which claims priority to U.S. Provisional Patent Application Ser. Nos. 60/812,632 filed Jun. 12, 2006, and 60/750,786 filed Dec. 16, 2005, the complete disclosures of which are incorporated herein by reference.

### FIELD OF THE INVENTIONS

The inventions relate to solid and liquid compositions that produce disinfectant solutions containing chlorine dioxide when combined with an aqueous solution.

### BACKGROUND

Chlorine dioxide is a highly reactive yellowish-green gas that produces useful aqueous solutions in a number of applications such as disinfection, sterilization, and odor control. It is a potent antimicrobial agent, bleaching agent, and as a germicide has found increasing receptivity of its use in municipal and drinking water treatment, cooling towers, and food processing.

Recent regulatory approvals have led to growing acceptance of its use in reducing pathogens in food processing applications such as poultry chill water tanks, beef and pork carcasses washes, and raw agricultural commodities. Chlorine dioxide has many advantages over traditional chlorine-based biocides due to its greater selectivity towards bacterial cell membranes.

Chlorine dioxide inactivates microorganisms by oxidizing key components in proteins that regulate cell metabolism. Cell death is caused by a loss of permeability control and ultimately membrane potential. By oxidizing these specific protein components, chlorine dioxide is reduced to inactive byproducts. The chemical reaction pathway prevents the formation of free chlorine, trihalomethanes (THMs) or haloacetic acids.

However, several drawbacks have limited the implementation of chlorine dioxide where its superior safety and environmental profile would benefit a wide variety of industries.

The prior art describes methods to produce chlorine dioxide from dry compositions such as tablets, powders, and briquettes. The prior art has been extensively covered in published U.S. patent appl'n ser. No. 2004/0135116, which is incorporated herein by reference.

U.S. Pat. No. 6,602,442 describes a “dry composition” comprising lithium hypochlorite, sodium chlorite, and sodium hydrogen sulfate. Although this mixture was found to very soluble and rapidly yield chlorine dioxide upon addition to water, a substantial amount of chlorine gas is undesirably released since chlorine is produced above its solubility in water. See column 2, line 55-56 of this patent. Furthermore, the stability of the dry mixture is limited, especially in high humidity environments. These limitations preclude the addition of large amounts to water since the excess chlorine production would cause the mixture to “flash”.

U.S. Pat. No. 7,087,190 describes a composition for the generation of chlorine dioxide including at least one non-iodo interhalide, polyhalide or salt having the formula: BrₘClₙFₒXₚ, wherein m=0-3, n=0-4, o=0-3, p=0-2, X is a cationic moiety and with the provisos that m+n+o, cannot be <2.

U.S. patent application 2006/0216223 A1 describes a process for generating aqueous chlorine dioxide solutions by adding a solid phase alkali metal chlorite, a solid phase acid, and a solid phase oxidizing agent to an aqueous solution, wherein the solid phase acid has a pKa less than 4. Compositions for producing chlorine dioxide solutions are also disclosed.

Published U.S. patent appl'n ser. No 2004/0135116 and U.S. Pat. No. 6,699,404 describe solid chlorine dioxide releasing “massive bodies,” which comprise a mixture of granular particulate ingredients where the size of the particles is substantially smaller than the size of the massive body. The massive body is formed from the mixture of particulate ingredients by compression and is essentially a large tablet. The tablets release chlorine dioxide and free chlorine when added to liquid water. The patent claims a composition of a chlorite salt, an acid source, and free halogen source, with the preferred composition being sodium chlorite, sodium bisulfate, and the sodium salt of dichloroisocyanuric acid.

Published US 2005/0249658 B2 describes a solid chlorine dioxide releasing composition having increased temperature stability. The increased temperature stability is provided by using a combination of two chlorine-releasing agents: sodium dichloroisocyanurate and a hypochlorite salt. The application also teaches that using an acidulant with a pKa of 2.8-6.0 further enhances temperature stability.

Furman and Margeum (Inorganic Chemistry 1998, 37, pages 4231-4327) discuss the rate of oxidation of chlorite ion by hypobromous acid. They describe the reaction as first-order and acid assisted in the presence of phosphate or carbonate buffers. They indicated the presence of a steady-state intermediate HOBrClO₂ that very rapidly reacts by two competing pathways: in one path reacts to form ClO₂ and bromide ions. In the other path OH⁻ reacts to form ClO₃ and bromide ions.

A halogen-enhanced oxidizing composition and a solvent activated reactor are described in published applications US 2005/0155936A1 and US 2006/0013751 A1. Solid oxidizing compositions including chlorine dioxide are described. The application describes the use of a metal chlorite, oxidizing agent, and chloride salt to produce chlorine dioxide.

US 2006/0016765 A1 and US 2006/0018940 A1 describe a sulfur-containing oxy-acid compound and precursors for generating chlorine dioxide. The preferred composition is sodium chlorite, potassium monopersulfate, and magnesium chloride. US 2006/0016765 A1 describes a composition for producing chlorine dioxide comprising an active oxygen compound and a chlorine dioxide-generating compound. See paragraph 23. Preferred active oxygen compounds are sulfur-containing oxy-acid compounds. See paragraph 26. Suitable precursors for producing chlorine dioxide include chlorite salt, alkali metal salt or alkaline earth halide salt. See paragraph 26. The preferred composition is sodium chlorite, potassium monopersulfate, and magnesium chloride. This publication discloses a large list of metal halides, which can be added as a “catalyst to speed up generation of chlorine dioxide” of which zinc bromide is listed. See paragraphs 42. However, the zinc bromide was used at a pH of 4.1. See Table 1. Furthermore, the publication teaches that calcium bromide at a pH of 3.7 is a “comparative example C,” i.e. does not work, and it gives an orange color which may interfere with results, thus, teaching away from using calcium bromide. See Table 1. Furthermore, this publication does not teach forming hypobromous acid at a pH of 5-9 or the many advantages thereof.

U.S. Pat. No. 6,303,038 discloses a water soluble dialkylhydantion and a source of bromide ion which are added to a body of water needing sanitization. This is followed by contacting the body of water with an oxidizing agent, which creates biocidal species in situ in the body of water. This patent does not however disclose the production of chlorine dioxide or using hypobromous acid to convert the chlorite ion to chlorine dioxide.

The benefits of using hypobromous acid to convert chlorite anions to chlorine dioxide are further disclosed in my co-pending published U.S. Patent Application No. 2008/0299161. However it has been discovered that when a solution is produced comprising relatively high concentration of chlorine dioxide, a competing reaction between hypobromous acid and chlorine dioxide results in the decomposition of chlorine dioxide, suppression of the solution's pH, and formation of bromine gas.

This decomposition of chlorine dioxide can be problematic. For example, when either tablet or powder compositions, such as in a flow-through feeder, in particular a brominator, are used to generate chlorine dioxide, the solution discharged from the flow-through system appears orange to brown in color, and the chlorine dioxide concentration is substantially lower than expected. In other experiments using tablets dissolved in water, is was noticed that the characteristic greenish-yellow color of chlorine dioxide, which formed quickly, slowly degraded over a period of hours to days to an orange-brown color with bromine-like odor accompanied by large drop in the pH (ie. from pH 7.5 to pH 3.0). It was also noticed that exposure to sunlight increased the rate of the chlorine dioxide decomposition.

### SUMMARY OF THE INVENTIONS

An objective is to prevent or reduce the decomposition of chlorine dioxide when a solution is produced comprising relatively high concentration of chlorine dioxide due to competing reactions between hypobromous acid and chlorine dioxide.

Another objective is to provide a stable solution of chlorine dioxide resistant to degradation by sunlight and chemical oxidation.

A further objective is to provide a composition present in solid or liquid form to yield chlorine dioxide on demand.

Another objective is to provide a means of chemical modification for preventing the undesirable competing reaction yielding suppression of pH and bromine gas formation.

A further objective is to provide a chlorine dioxide solution with extended disinfectant properties beyond which would normally be useful.

Another objective is to provide a composition that attains a near maximum theoretical conversion of sodium chlorite to chlorine dioxide.

These objectives and other objectives are obtained by a solid composition for producing a stabilized solution comprising chlorine dioxide and free bromine, the composition comprising: a solid source of oxidizing agent; a solid source of hypobromous acid; a solid source of chlorite; a solid source of free bromine stabilizer, wherein the solid inorganic source of hypobromous acid and solid source of chlorite are present in an amount such that the pH of a solution formed from dissolving the composition in water is from 5 to 9, measured at 25° C. at a concentration of 1 g of active ingredients of the composition per 100 ml of water, the active ingredients being the total weight of the solid inorganic source of hypobromous acid and the solid source of chlorite.

The objectives and other objectives are also obtained by a method of producing chlorine dioxide comprising adding a composition comprising a solid inorganic source of hypobromous acid, a solid source of chlorite and a free bromine stabilizer, wherein the solid inorganic source of hypobromous acid and solid source of chlorite are present in an amount such that the pH of a solution formed from dissolving the composition in water is from 5 to 9, measured at 25° C. at a concentration of 1 g of active ingredients of the composition per 100 ml of water, the active ingredients being the total weight of the solid inorganic source of hypobromous acid and the solid source of chlorite, to water such that hypobromous acid is produced from the solid inorganic source of hypbromous acid and the hypobromous acid is reacted with chlorite produced from the solid source of chlorite to form chlorine dioxide.

The objectives and other objectives are further obtained by a multi-part system for producing chlorine dioxide on demand comprising an aqueous solution containing sodium chlorite, sodium bromide, and a free bromine stabilizer and a solid comprising at least potassium monopersulfate.

The objectives and other objectives are obtained by a method for producing chlorine-dioxide comprising combining an aqueous solution containing sodium chlorite, sodium bromide, and a free bromine stabilizer with a solid comprising at least potassium monopersulfate to thereby produce chlorine dioxide.

### BRIEF DESCRIPTION OF THE DRAWINGS

FIG. 1 illustrates a relative equilibrium for the generation of chlorine dioxide using hypobromous acid generated by the reaction between monopersulfate anion and sodium bromide; and

FIG. 2 illustrates a relative equilibrium of reactants and products for a system like that illustrated in FIG. 1, with the inclusion of an effective amount of stabilizer exemplified by 5,5-Dimethylhydantoin (DMH)

### DETAILED DESCRIPTION OF THE INVENTIONS

### A. Compositions of the Inventions

The compositions comprise a solid source of source of hypobromous acid, a solid source of chlorite and a free bromine stabilizer. The active ingredients of the compositions are selected and present in amounts such that the pH of a solution formed from dissolving the composition in water is from about 5 to about 9, preferably from about 5.5 to about 8.5, and more preferably about 6 to about 8, and most preferably about 6.5 to 7.5, measured at 25° C. at a concentration of 1 g of active ingredients of the composition per 100 ml of water. The active ingredients being the solid source of hypobromous acid and the solid source of chlorite.

The solid source of chlorite comprises an alkali or alkali earth metal chlorite. The solid source of hypobromous acid comprises an oxidizing agent and a solid bromide releasing compound.

The alkali or alkali earth metal chlorite can be any as desired, such as sodium, potassium, lithium, calcium, zinc, or magnesium chlorite, which are commercially available.

The compositions typically contain from about 10 to about 90% of solid source of hypobromous acid and about 10 to about 90% of solid source of chlorite. All percentages are weight percentage based on the total weight of the composition unless otherwise stated.

Preferred composition comprises from about 10 to about 90% of sodium chlorite, about 5 to about 90% of sodium bromide, and about 5 to about 60% of potassium monopersulfate. More preferably, the composition comprises about 20 to about 60% sodium chlorite, about 10 to about 40% of sodium bromide, and about 10 to about 50% of potassium monopersulfate. All percentages are weight percentage based on the total weight of the composition unless otherwise stated.

An alternative composition comprises from about 10 to 90% of sodium chlorite and about 1 to 80% s-triazine ring derivates as shown below:

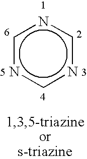

where halogened substitutions at the three nitrogen positions are as follows: 1=Br; 3=H, Br or Cl; and 5=H, Br, or Cl. Alkali and alkali earth metal salts of the above halocyanurates are suitable. Preferred brominated triazines are the sodium salts of bromochloroisocyanuric acid (CAS #20367-88-8) and bromoisocyanuric acid (CAS#164918-61-0) and both are commercially available.

Oxidizing agents are typically acidic in nature. Thus, preferably the amount of oxidizing agent is from about 5 to about 50%, more preferably from about 10 to about 40%, and most preferably from about 10 to about 30% of the composition, based on the total weight of the oxidizing agent, chlorite and bromide-releasing compound.

Suitable examples of oxidizing agents include alkali and alkali earth metal persulfates, monopersulfates, and ammonium persulfate; alkali and alkali earth peroxides such as lithium, sodium, potassium, calcium, zinc, or magnesium peroxide, urea peroxide, percarbonates, persilicates, perphosphates and their metal salts, and hydrogen peroxide. The preferred oxidizing agent is potassium monopersulfate.

Other example of peroxides other than hydrogen peroxide include dialkylperoxides, diacylperoxide, performed percarboxylic acids, organic and inorganic peroxides, and/or hydroperoxides. Suitable organic peroxides/hydroperoxides include diacyl and dialkyl peroxides such as dibenzoyl peroxide, t-butyl hydroperoxide, dilauroyl peroxide, dicumyl peroxide, and mixtures thereof.

Suitable peroxy-acids for use in the compositions include diperoxydodecandioic acid (DPDDA), magnesium perphthalic acid, perlauric acid, perbenzoic acid, diperoxyazelaic acid and mixtures thereof.

Suitable examples of bromide salts include alkali and alkali earth metal bromides such as sodium bromide, lithium bromide, potassium bromide, magnesium bromide, ammonium bromide, zinc bromide, calcium bromide, and aluminum bromide.

Other examples of bromide salts include mixtures of bromine-rich salts commonly found in nature such as the Dead Sea salts and brines. These contain bromide salt in combination with other common salts such as sodium chloride, potassium chloride, magnesium chloride, zinc chloride, sodium sulfate, potassium sulfate, magnesium sulfate, sodium iodide, potassium iodide, and the like. Any combination of salts can be used as the bromide source.

Other exemplary combinations include: a bromide salt combined with a chloride or iodide salt. For example: bromide salt+sodium, potassium, lithium, magnesium, calcium, zinc, ammonium, or aluminum chloride. Or for example an iodide salt of sodium, potassium, lithium, calcium, magnesium, zinc, ammonium, or aluminum can also be used. All can be used individually or in combination in the current invention.

The source of the halide salts required to produce the hypohalous acid can also be present in the water which is to be treated. For example, seawater which consists of about 3.5% salt can be used as the sole source of the halide salt.

The chlorine dioxide releasing composition then can be reduced to two parts: chlorite source and the oxidizing agent. This unexpected result was found when adding sodium chlorite and potassium monopersulfate to seawater (See Experimental Example 6).

Also suitable are bromide-releasing compound in which the bromide ion is a salt of a suitable organic cation, such as for example, but not limited to ammonium bromide, alkylammonium bromide, dialkylammonium bromide, trialkylammonium bromide, wherein said alkyl radicals are independently selected from straight chain or branched aliphatic, aromatic, or aryl hydrocarbon radicals of between 1 to about 24 carbon atoms.

Also suitable as a bromide-releasing compound are bromide ion exchange materials. That is materials able to exchange a bromide ion in the presence of the more common chloride ion in aqueous solution, and which are typically water insoluble polymeric and mineral matrixes preloaded with high bromine ion content.

Ideally the salt should form hypobromous acid during oxidation with potassium monopersulfate. Some salts or mixtures of salts will form a combination of hypobromous, hypochlorous, and/or hypoiodous acid. Hypochlorous acid is also a powerful oxidizing agent and will convert additional bromide salt to hypobromous acid.

Other forms of bromine-releasing compounds may also be used. The below compounds provide free bromine and the corresponding hypobromous acid when added to liquid water. They can also serve as oxidizing agents to convert bromide salts to free bromine. Also suitable to provide free bromine is liquid bromine in elemental form. This is a dark liquid at room temperature and standard pressure.

### Hypohalite-Generating Compound

A hypohalite-generating compound can provide the oxidation potential to oxidize sodium chlorite to chlorine dioxide. A hypohalite-generating compound can also produce free bromine from bromide ions in aqueous solution. Suitable compounds for providing the free available halogen concentration are hypochlorite-generating or hypobromite-generating compounds. These compounds must be at least partially or fully water soluble and generate an active halogen ion (ie. HOCl, HOBr, OCl⁻, OBr⁻) upon dissolving in water. Thus, the hypohalite-generating compound can be considered as both the oxidizing agent and the bromide-releasing agent.

Any of the following representative sources or mixtures thereof include alkali and alkali earth metal hypobromite salts such as: lithium hypobromite, sodium hypobromite, potassium hypobromite, calcium hypobromite, magnesium hypobromite, and zinc hypobromite.

Also suitable hypochlorite-generators are chlorinated trisodium phosphate, chlorinated trisodium polyphosphate, and chlorinated trisodium phosphate dodecahydrate, and mixtures thereof.

Any of the following representative sources or mixtures thereof including alkali and alkali earth metal hypochlorite salts such as lithium hypochlorite, sodium hypochlorite, potassium hypochlorite, calcium hypochorite, magnesium hypochlorite, and zinc hypochlorite are also suitable.

Additional suitable hypobromite-generating organic compounds include N-bromophthalamide, N,N-dibromodimethylhydantoin, N,N-dibromodiethylhydantion, N,N-dibromodimethylglycoluracil, dibromotriethylene-diamine dihydrogenchloride, and mixtures thereof.

Suitable hypochlorite-generating compounds include: Trichlorocyanuric acid, dichlorocyanuric acid, mono-chlorocyanuric acid, sodium dichloroisocyanurate (dihydrate and anhydrous), and potassium dichloroisocyanurate.

Also suitable hypochlorite-generating compounds including but not limited to N,N-dichloro-s-trizinetrione, N-chlorophtalamide, N-dichloro-p-toluene sulfonamide, 2,5-N,N-dichloroazodicarbonamidine hydrochloride, NNNN-tetrachloroglycoluracil, N,N-dichloroyl,N,N,N-trichloromelamine, N-chlorosuccinimide, methylene-bis(1-chloro-5,5,-dimethyethylhydantoin), 1,3-dichloro-5-methyl-5-isobutylhydantoin, 1,3,dichloro-5-n-amylhydantoin, 1,3-dichloro-5,5-dimethylhydantoin, 1,4-dichloro-5,5-diethylhydantoin, 1-1 monochlor-5,5-dimethylhydantoin, sodium-para-toluenesulfochloramine, dichlorosuccinamide, 1,3,4,6-tetrachlorogylcouracil, potassium and sodium salts of chloroisocyanuric acid, dichloroisocyanuric acid, potassium and sodium salts of N-brominated and N-chlorinated succinimide, malonimide, phthalimide and naphthalimide, and mixtures thereof.

Halohydantoins, such as 1-bromo-3-chloro-5,5-dimethylhydantion (BCDMH), 1-bromo-3-chloro-5-methyl-5-ethyl-hydantoin (BCEMH), 1,3-dibromo-5,5-dimethylhydantoin (DBDMH), 1,3-dibromo-5-methyl-5-ethyl-hydantoin (DBEMH) are suitable.

Other suitable N-haloamines are trichloromelamine, tribromomelamine, dibromo- and dichloro-dimethyhydantoin, chlorobromo-dimethylhydantoin, N-chlorosulfamide (haloamide), chloramines (haloamine), and mixtures thereof.

Also suitable are partially chlorinated and brominated compounds, including N-bromo-N-chlorodimethylhydantion, N-bromo-N-chlorodiethylhydantoin, N-bromo-N-chlorodiphenylhydantoin, N-bromo-N,N-dichloro-dimethylglycouracil, N-bromo-N-chlorosodium cyanurate, bromochlorotriethylenediamine dihydrogenchloride and mixtures thereof.

### Free Bromine Stabilizer

The free bromine stabilizer comprises a compound: containing nitrogen chemically bonded to at least one carbon comprising a carbonyl group; resistant to oxidation in a solution comprising hypobromous acid and chlorine dioxide; and imposes a stabilizing effect to free bromine by forming a complex between the nitrogen and the Br⁺ portion of the free bromine.

In the present invention, dialkylhydantions are preferred. The alkyl groups of the dialkylhydantion may be the same or different, and are both bound to the carbon atom at the 5-position of the hydantion ring. Suitable alkyl groups include methyl, ethyl, isopropyl, tert-butyl, methylcyclopentyl, cyclohexyl, and the like. Preferred dialkylhydantions are 5-ethyl-5-methyl-hydantion and 5,5-dimethylhydantoin.

Examples of free bromine stabilizer(s) include but are not be limited to: 5,5-dimethylhydantoin; cyanuric acid, sulfamic acid, N-succinimide, glycoluril; and glutarimide.

As used herein “free bromine” comprises at least one of: hypobromous acid (HOBr); and hypobromite ion (OBr⁻) resulting from the hydrolysis of Br⁺ in an aqueous solution. The Br⁺ portion of the free bromine may result from the hydrolysis of bromine gas in water, or oxidation of bromide anions to form Br₂ or HOBr.

As used herein “stabilized” refers to the presence of stabilizer in a solution comprising at least chlorine dioxide and free bromine, wherein the concentration of stabilizer is sufficient to provide at least some portion of stabilized bromine. To exemplify without limiting stabilizers, a solution is stabilized by DMH when sufficient DMH is present to convert at least some portion of free bromine to a bromine-DMH complex such as bromodimethylhydantoin.

As used herein “reactants” represent any of the components in the composition that produce at least one of chlorine dioxide and hypobromous acid. For example, if hypoberomous acid is generated by the reaction between potassium monopersulfate and sodium bromide, potassium monopersulfate and sodium bromide are reactants for producing hypobromous acid. If chlorine dioxide is produced by reacting hypobromous acid with sodium chlorite, then sodium chlorite, sodium bromide and potassium monopersulfate are all reactants.

As used herein “an effective amount” refers to the addition of a free bromine stabilizer sufficient to achieve the desired level of chlorine dioxide stability. Depending on the desired level of chlorine dioxide stability, the free bromine stabilizer can be included in substoichiometric, stoichiometric or suprastoichiometric concentrations based on the maximum concentration of free bromine that can be produced in the stabilizer treated solution.

Both powder and agglomerated compositions that incorporate: an oxidizer, chlorite anion, and bromide anion have been shown to be extremely effective at generating chlorine dioxide. However, in highly concentrated solution the stability of the chlorine dioxide can be compromised by the development of a competing reaction that decomposes the chlorine dioxide.

There are a series of reactions that take place in the oxidant, bromide, chlorite system. In the exemplified reaction for producing hypobromous acid, monopersulfate was used to illustrate the process. However, other oxidants, such as those described above, can also be used to activate the bromide ion to produce HOBr. Relevant reactions are proposed as follows:
a. Methods of Generating HOBr
b. HSO₅⁻+Br⁻>>>HOBr+SO₄⁼
2. HSO₅⁻+2Br⁻+H⁺>>>Br₂+SO₄⁼+H₂O
a. Br₂+H₂O<<<>>>HOBr+HBr
i. ClO₂ Formation
ii. HOBr+2NaClO₂>>>2ClO₂+NaBr+NaOH
1. Br₂+2NaClO₂>>>2ClO₂+NaBr
a. Decomposition of ClO₂↑
2. HOBr+ClO₂↑>>>ClO₃⁻+[Br⁺+H+]
a. Formation of Bromine Gas
3. [Br⁺+H⁺]+Br⁻<<<>>>Br₂↑+H⁺
4. DMH Stabilization of ClO₂ in HOBr System
iii. C₅H₈N₂O₂+Br⁺<<<>>>C₅H₇BrN₂O₂ (BDMH)
iv. C₅H₇BrN₂O₂+Br⁺<<<>>>C₅H₆Br₂N₂O₂ (DBDMH)

Of significant importance is the equation for the Decomposition of ClO₂. As illustrated in the testing section, several changes occur in rapid succession. The pH begins to decline. This is followed by a period of sustained or increase chlorine dioxide concentration followed by a dramatic drop in chlorine dioxide concentration. There is also the noticeable color change that corresponds to the pH nearing that when bromine gas is formed and stabilized. Finally, when the pH is increased there is no reactivation of chlorite by reaction with hypobromous acid to produce chlorine dioxide. Therefore it is evident the chlorine dioxide is not reduced to chlorite anions, but rather oxidized to chlorate anions.

The equation proposed for the decomposition of chlorine dioxide by reaction with hypobromous acid supports the formation of: chlorate anion (ClO₃⁻); bromine ion (Br⁺); and hydrogen ion (H⁺). The byproducts are then involved in the next proposed reaction whereby bromine cation (Br⁺) reacts with bromide anion (Br⁻) under acidic conditions resulting in the formation of stable bromine gas.

The addition of a free bromine stabilizer such as 5,5-Dimethylhydantoin can be used to extend the stability of the solution until such time the solution is diluted thereby offsetting the decomposition reaction, or altogether stabilizing the concentrated solution by substantially reducing the concentration of hypobromous acid. Since the equilibrium between the concentration of free bromine and the concentration of stabilized bromine is a function of the DMH concentration, suprastoichiometric levels can substantially reduce the presence of free bromine in the solution and effectively stabilize the chlorine dioxide and residual oxidants.

The free bromine stabilizer can be combined with the composition, be added to the solution either before or after addition of the composition, or be supplied by the water used to dissolve the composition such as in the case of swimming pool water comprising DMH.

If desired, the composition can be in the form of more than one part, and be present in solid or liquid media. For example, a solution containing metal chlorite, metal bromide, and DMH with the pH buffered between pH 8-pH 11 can be produced and stored for future use—further known as Solution A. Chlorine dioxide can be produced on demand by adding an oxidizer such as monopersulfate, in powder or tablet form to a premeasured amount of Solution A. The result is the rapid production of pH neutral chlorine dioxide. The free bromine stabilizer may be present in either part of the formulation (soluble or in solid form).

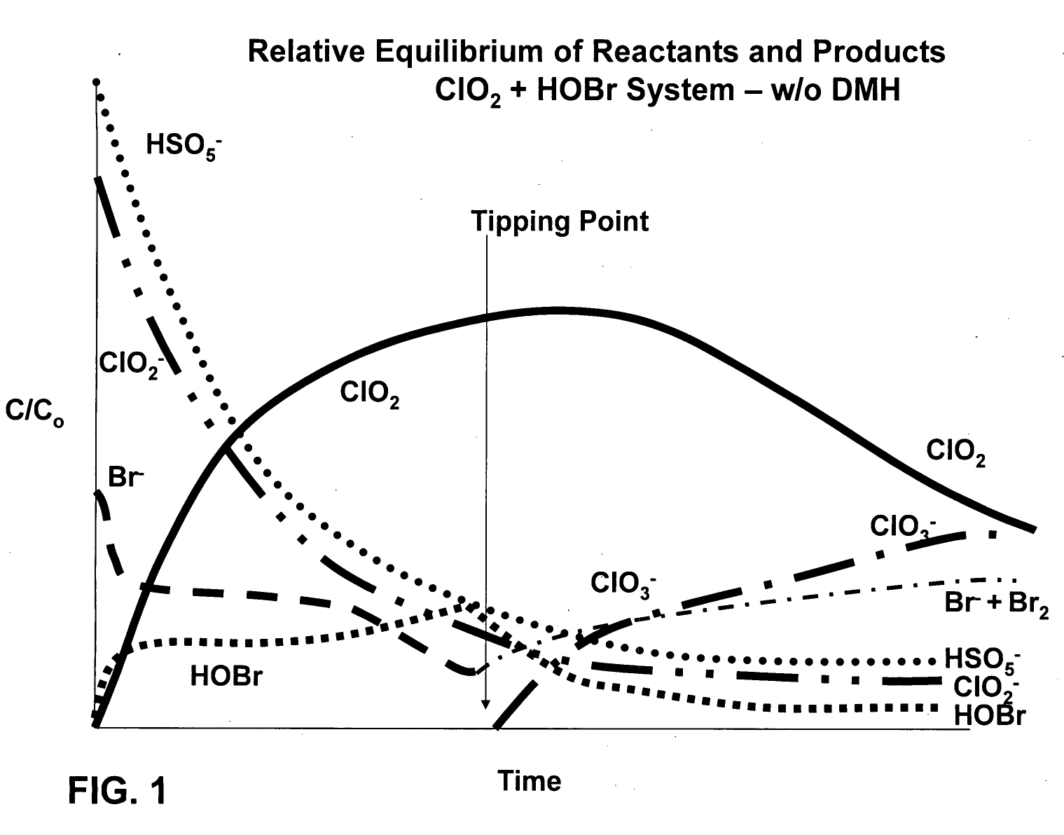

FIG. 1 shows a relative equilibrium for the generation of chlorine dioxide using hypobromous acid generated by the reaction between monopersulfate anion and sodium bromide. The graph illustrates the initial conversion of bromide to hypobromous acid with a corresponding reduction in monopersulfate anion and chlorite anion, with an increase in chlorine dioxide. As the reactions proceed the concentration of chlorite anions is overtaken by the concentration of chlorine dioxide. The declining chlorite anion concentration results in a decrease in the reduction of hypobromous acid to bromide anions, resulting in an increase in hypobromous acid concentration. The point in time when the generation of chlorine dioxide begins to be overshadowed by the decomposition of chlorine dioxide is designated the “tipping point”. At the tipping point, chlorine dioxide can still be generated however it is also being decomposed at an increased rate. The decomposition of chlorine dioxide results in the suppression of pH, formation of bromine gas, and a chlorine dioxide decomposition product that cannot be converted back to chlorine dioxide by the hypobromous acid system (chlorate anion).

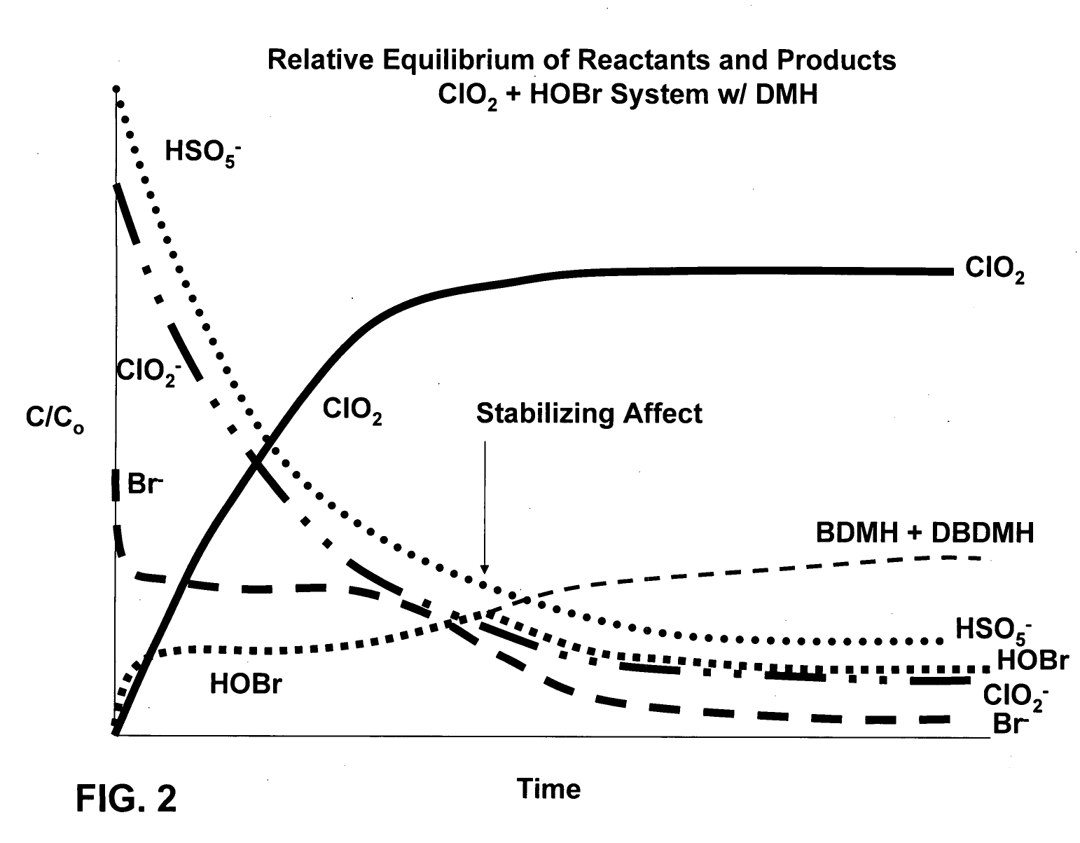

FIG. 2 shows a proposed relative equilibrium of reactants and products for a system like that illustrated in FIG. 1, with the inclusion of an effective amount of stabilizer exemplified by 5,5-Dimethylhydantoin (DMH). In the illustration, the stabilizing affects of DMH prior to the “Stabilizing Affect” is not shown. As the hypobromous acid concentration increases due to the declining chlorite anion and corresponding reduction of hypobromous acid to bromide anion, the benefit contributed by the stabilizing affect of the DMH becomes apparent. Instead of a drop in chlorine dioxide concentration and other desirable oxidants, the chlorine dioxide concentration is stabilized and residual hypobromous acid is converted to Bromodimethylhydantoin (BDMH) and/or Dibromodimethylhydantoin (DBDMH).

In some instances it may not be desirable to use a free bromine stabilizer to prevent the undesirable competing reaction. If a substoichiometric amount of oxidizer, such as monopersulfate, is employed the suppression of pH and color change will be avoided. However, the yield of chlorine dioxide would be substantially reduced from what might be obtained with the use of free bromine stabilizer.

The compositions can contain other ingredients as desired. For example, if the composition is formed into a tablet, common ingredients such as binders, mold release agents, compression aids, tablet lubricants, swelling agents, carrier materials, fillers, and surfactants can optionally be added to the composition.

The dry components used to produce chlorine dioxide exemplified by potassium monopersulfate, sodium chlorite, sodium bromide and/or DMH may be coated to provide additional protection from the elements such as relative humidity. Magnesium carbonate light sold under the trade name Elastocarb® by Akrochem Corporation and Fumed Silica sold under the trade name Cab-O-Sil® by Cabot Corporation provide improved flow characteristics and environmental stability for powders and improved processing and stability of agglomerated composition such as granules and tablets. Coating may also reduce the NFPA and DOT rating by reducing their reactivity.

The coating can be applied by mixing the coating and the dry component in a ribbon mixer, tumbling mixer, spray coating or any number of existing coating methods.

The compositions of the invention can further benefit by coating the at least one of the reactants using a process Magnetic Assisted Impact Coating (MAIC), available by Aveka, Inc. The coatings applied using MAIC provide an adhered coating that is not removed by typical handling such as in the case of tablet compression, packaging and handling.

### EXAMPLES

Samples of compositions were prepared in powder and tablet form and applied to 500 ml of water with slow mixing using a magnetic stirrer to compare the chlorine dioxide stability. Samples 1 through 3 utilized high concentrations of NaBr to accelerate the generation of chlorine dioxide and induce formation of excess hypobromous acid.

Sample 1 illustrates that 3-minutes into the reaction the chlorine dioxide approach it's maximum concentration. By 4-minutes a distinct color change was evident, and by 5-minutes of lapsed time, the sample had taken on an orange color.

TABLE 1

| | Lapsed Time | | |
|---|---|---|---|
| Sample #1 | (Min) | ClO₂ (ppm) | Color |
| NaClO₂ 1.0 gm | 2 | 655 | Yellow |
| KMPS 1.5 gm | 3 | 659 | Yellow |
| NaBr 0.9 gm | 4 | 618 | slight orange |
| | 5 | 500 | orange |

Sample 2 illustrates the addition of 0.2 grams of 5,5-Dimethylhydantoin (DMH) to the powder mixture was sufficient to significantly stabilize the chlorine dioxide. It is also apparent the addition of the DMH slowed the initial development of chlorine dioxide. However, the maximum concentration of chlorine dioxide produced was comparable to the sample 1 that did not contain DMH.

TABLE 2

| | Lapsed Time | | |
|---|---|---|---|
| Sample #2 | (Min) | ClO₂ (ppm) | Color |
| NaClO₂ 1.0 gm | 2 | 535 | Yellow |
| KMPS 1.5 gm | 5 | 597 | Yellow |
| NaBr 0.9 gm | 10 | 620 | Yellow |
| DMH 0.2 gm | 25 | 649 | Yellow |
| | 40 | 649 | Yellow |

Sample 3 included only 0.1 gm DMH along with the addition of sodium bisulfate. This combination increased the concentration of chlorine dioxide generated and slowed the eventual decomposition of chlorine dioxide to a rate slower than that of sample 1, but not as stable as sample 2 that had twice the concentration of DMH.

TABLE 3

| | Lapsed Time | | |
|---|---|---|---|
| Sample #3 | (Min) | ClO₂ (ppm) | pH |
| NaClO₂ 1.0 gm | 1 | n/a | 5.82 |
| KMPS 1.5 gm | 2 | 702 | n/a |
| NaBr 0.9 gm | 3 | n/a | 6.79 |
| DMH 0.1 gm | 5 | 725 | 7.13 |
| NaHSO₄ 0.2 gm | 10 | 717 | 7.20 |
| | 22 | 685 | 7.05 |

Samples 4 through 6 used lower levels of sodium bromide (NaBr) and where applicable, lower concentrations of DMH (sample 5 and 6).

Sample 4 shows the chlorine dioxide concentration approaches it's maximum level after about 7-minutes. At 9-minutes the pH approaches it's maximum level, then begins declining. By 12-minutes the chlorine dioxide concentration has noticeably dropped along with the declining pH. By 13-minutes the color of the solution begin turning noticeably darker. By 15-minutes the solution has turned orange in color and the pH has precipitously dropped. By 15.25-minutes, the pH drops to 2.49.

TABLE 4

| | Lapsed Time | | |
|---|---|---|---|
| Sample #4 | (Min) | ClO₂ (ppm) | pH |
| NaClO₂ 1.0 gm | 1 | n/a | 2.43 |
| KMPS 1.5 gm | 2 | 545 | 2.70 |
| NaBr 0.2 gm | 3 | n/a | 4.40 |
| NaHSO₄ 0.2 gm | 4 | 677 | 5.55 |
| | 5 | n/a | 6.17 |
| | 7 | 763 | 6.68 |
| | 9 | n/a | 6.73 |
| | 9.5 | 764 | 6.64 |
| | 11 | n/a | 6.44 |
| | 12 | 740 | 6.25 |
| | 13 | color change | 6.06 |
| | 14 | n/a | 5.78 |
| | 15 | Orange | 4.67 |
| | 15.25 | n/a | 2.49 |

Sample 5 is a replica of sample 4 except with the addition of 0.02 grams of DMH. As previously illustrated, the addition of DMH reduces the rate of chlorine dioxide generation, however the maximum chlorine dioxide concentration is higher and the solution remains stable well after the sample without DMH began decomposing the chlorine dioxide.

TABLE 5

| | Lapsed Time | | |
|---|---|---|---|
| Sample #5 | (Min) | ClO₂ (ppm) | pH |
| NaClO₂ 1.0 gm | 1 | n/a | 2.71 |
| KMPS 1.5 gm | 2 | 522 | 2.70 |
| NaBr 0.2 gm | 4 | n/a | 3.60 |
| DMH 0.03 gm | 5 | 655 | 4.90 |
| NaHSO₄ 0.2 gm | 9 | n/a | 6.18 |
| | 10 | 738 | 6.33 |
| | 15 | 766 | 6.76 |
| | 17 | ≧770 | 6.78 |

Sample 6—A clear plastic column with end caps comprising a tube connector was fitted with a cotton mesh on the bottom followed by a plastic mesh packing that reach about half way up the column. A powder composition was prepared by crushing 1.00 grams of sodium chlorite, adding in 0.2 grams of fumed silica, mixing, then adding the remainder of components with mixing after each addition. The mixture was added to the column followed by addition of more plastic mesh to fill the remaining head space. The top end cap with the tube connector was inserted onto the end of the column. The column was secured to a clamp mounted onto a stand.

A ⅜ in. plastic tube connected at the discharge of a 1-gph LMI metering pump was inserted onto the bottom end cap by slipping the tube over the tube connector, then removed to allow priming of the pump. A second length of tube was inserted onto the top end cap by slipping the tubing over the tube connector. The free end of the tubing was inserted into a beaker containing 3500 ml of water so that the tube was flush against the bottom of the beaker.

A funnel was connected to the top of the supply tubing going to the suction of the LMI metering pump. The funnel was secured with a clamp by connecting the clamp to a stand. Water as added to funnel, the pump was turn on, and a priming valve was opened to remove air from the diaphragm of the pump. Once the supply tubing, pump, and discharge tubing were cleared of air, the discharge tubing from the pump was connected to the bottom of the column containing the reactants.

The funnel was topped of with 250 ml of water, which was enough to fill the column (40 ml) and allows sufficient flushing of the column and column discharge tubing.

The pump was turned on and set at 50% output (approx 32 ml/min). When the water reached the reactants, yellow gas was formed which displaced air from the top of the column and discharge tubing. Shortly later a solution of chlorine dioxide was observed discharging through the column's outlet tubing and flowing into the bottom of the 3500 ml beaker of water. After approximately 5 minutes, the pump was turned off, and the seal at the top of the column was carefully broken to allow air into and displace the remaining liquid allowing it to flow to the receiving beaker. A total of about 3650 ml of solution was in the receiving beaker.

The solution in the beaker was mixed to produce a homogenous solution. Three consecutive samples were taken to obtain and confirm the chlorine dioxide concentration using a Hach Spectrophotometer. The results of the test showed 117 ppm of chlorine dioxide. The chlorite conversion was calculated to be 72.14%.

TABLE 6

| Sample #6 | ClO₂ in 3650 ml |
|---|---|
| NaClO₂ 1.0 gm | 117 ppm |
| KMPS 1.5 gm | |
| NaBr 0.2 gm | |
| DMH 0.02 gm | |
| NaHSO₄ 0.1 gm | |

Sample 7-10 describe various compositions compressed into tablets. Tablets were compressed on a Carver laboratory press at 25,000 lbs. The tablets were then added to various amounts of water and chlorine dioxide concentration was measured after 15 minutes. The conversion yield of sodium chlorite to chlorine dioxide was calculated from the final concentration of chlorine dioxide and amount of sodium chlorite present in the tablet. pH values were also measured.

TABLE 7

| Sample# | NaClO2 (g) | NaBr (g) | KMPS (g) | DMH % |
|---|---|---|---|---|
| 7 | 50 | 10 | 65 | 0 |
| 8 | 50 | 10 | 65 | 2 |
| 9 | 50 | 10 | 65 | 1 |
| 10 | 50 | 10 | 75 | 1.5 |

TABLE 8

| Sample | Tablet (g) | Water (L) | ClO2 ppm | Yield % | pH | Color |
|---|---|---|---|---|---|---|
| 7 | 10 | 2 | 872 | | 3.8 | Brown |
| 8 | 10 | 2 | 800 | 84.5 | 7.8 | Yellow |
| 9 | 10 | 2 | 859 | 90.7 | 7.6 | Yellow |
| 9 | 11 | 3 | 649 | 93 | 7.7 | Yellow |
| 10 | 10 | 2 | 842 | 98.2 | 7.4 | Yellow |
| 10 | 10 | 8 | 212 | 98.8 | 7.5 | Yellow |
| 10 | 10 | 6 | 275 | 96.2 | 7.5 | Yellow |

Sample 11

The composition was divided into two parts as below:

Part A

| | |
|---|---|
| NaClO2 | 10 grams |
| NaBr | 2 grams |
| DMH | 1 gram |
| Total | 13 grams |

Part B

| | |
|---|---|
| KMPS | 4 grams (made into a tablet as above) |

The following was added to 1 Liter of water:

| | |
|---|---|
| Part A | 2 grams |
| Part B | 4 grams |

| Time | 15 | 20 | 25 | 30 |
|---|---|---|---|---|
| Concentration | 616 ppm | 794 pmm | 931 ppm | 970 ppm |

Theoretical yield: 2 grams×0.77( 10/13 grams)=1.54 grams NaClO2

1.54 grams×0.82% purity/90 grams per mole=0.01403 moles theoretical yield

Actual results: 970 ppm×1000 ml=0.97 grams/67=0.01447 moles (this is a conversion of 103%). All read on Hach spectrometer as above.

The conversion of 103% is thought to have arisen due to a correction in the purity of sodium chlorite. If a purity of 85% is used then the calculation is adjusted:

1.54 grams×0.85% purity/90=0.01454 moles; then the conversion yield is 99.5%

Sample 12—Composition was Tested with Succinimide Stabilizer:

| | | |
|---|---|---|
| NaClO2 | 20 | g |
| NaBr | 4 | g |
| KMPS | 30 | g |
| Succinimide | 1 | g |

10 gram tablets made as above
10 g tablet into 2000 ml=730 ppm
10 g tablet into 3000 ml=478 ppm

| | | |
|---|---|---|
| NaClO2 | 20 | g |
| NaBr | 4 | g |
| KMPS | 30 | g |

10 gram tablet into 2000 ml=brown color

The results clearly demonstrate that the presence of stabilizers such as DMH and succinimide prevent the undesirable competing reaction described above. DMH substantially improved the overall efficiency of the reaction and facilitated higher conversion of chlorite to chlorine dioxide. It was also observed that the effects from other forms of degradation stimuli such as sunlight were largely mitigated in the “stabilized” chlorine dioxide solutions.

Sample 13—Composition Using Halocyanurate Derivate—Sodium Bromochlorisocyanuric Acid:

TABLE 9

| | | | | | | |
|---|---|---|---|---|---|---|
| NaClO2 (g) | 1 | 1 | 1 | 1 | 1 | 1 |
| BCCA (g) | 0.5 | 0.25 | 0.1 | 0.5 | 0.25 | 0.1 |
| Water (ml) | 1000 | 1000 | 1000 | 2000 | 2000 | 2000 |
| ClO2 (ppm) | 376 | 200 | 100 | 196 | 113 | 56 |

Calculate Yield Conversion:

1 g×0.8/90=0.0088 moles ClO2 theoretical conversion
376 ppm=0.376 grams/67=0.00561 moles actual result=63.6% yield

### Disinfection Efficacy Studies

Ten gram tablets were prepared from the following materials as above:

| | | |
|---|---|---|
| Sodium chlorite | 20 | gram |
| Sodium bromide | 4 | gram |
| Potassium monopersulfate | 26 | grams |

The tablets were challenged with microorganisms following Good Laboratory Practice methods and results are provided in Table 10:

TABLE 10

| | | Concen- | | Exposure | | |
|---|---|---|---|---|---|---|
| Organism | Method | tration | | time | | Result |
| Staph aureus | AOAC Use | 275 | ppm | 1 | minute | 0/40 |
| ATCC 6538 | Dilution | | | | | survivors |
| P aeruginosa | AOAC Use | 135 | ppm | 10 | minute | 0/60 |
| ATCC 15442 | Dilution | | | | | survivors |
| M terrae | TB surrogate, | 200 | ppm | 5 | minute | Killed > |
| ATCC 15755 | Rate of Kill | | | | | 7log₁₀ |
| | Test | | | | | |
| C sporogenes | AOAC | 1200 | ppm | 1 | hour | 0/40 |
| ATCC 3584 | sporicidal | | | | | survivors |
| | method | | | | | |

The results of the microbial challenge study show that the tablets had wide spectrum efficacy over several concentrations and exposure times.

While the claimed invention has been described in detail and with reference to specific embodiments thereof, it will be apparent to one of ordinary skill in the art that various changes and modifications can be made to the claimed invention without departing from the spirit and scope thereof.

I claim:

1. A solid composition for producing a stabilized solution comprising chlorine dioxide and free bromine, the composition comprising:
a solid source of oxidizing agent;
a solid source of hypobromous acid;
a solid source of chlorite;
a solid source of free bromine stabilizer, wherein the solid inorganic source of hypobromous acid and solid source of chlorite are present in an amount such that the pH of a solution formed from dissolving the composition in water is from 5 to 9, measured at 25° C. at a concentration of 1 g of active ingredients of the composition per 100 ml of water, the active ingredients being the total weight of the solid inorganic source of hypobromous acid and the solid source of chlorite.

2. A solid composition according to claim 1, wherein solid source of free bromine stabilizer comprises at least one selected from the group consisting of 5,5-dimethylhydantoin, glycoluril, glutarimide, cyanuric acid, or succinimide.

3. A solid composition according to claim 1, wherein the solid source of chlorite comprises a metal chlorite and the solid inorganic source of hypobromous acid comprises an oxidizing agent and a bromide salt.

4. A solid composition according to claim 3, wherein the oxidizing agent is at least one oxidizing agent selected from the group consisting of alkali and alkali earth persulfates, monopersulfates, alkali and alkali earth peroxides, urea peroxide, percarbonates, persilicates, perphosphates, and hydrogen peroxide.

5. A solid composition according to claim 3, wherein the bromide salt comprises at least one bromide salt selected from the group consisting of alkali and alkali earth metal bromides.

6. A solid composition according to claim 3, wherein the bromide salt comprises at least one selected from the group consisting of sodium bromide, lithium bromide, potassium bromide, calcium bromide, magnesium bromide, ammonium bromide, and aluminum bromide.

7. A solid composition according to claim 1, wherein the solid source of chlorite comprises an alkali or alkali earth metal chlorite and the solid inorganic source of hypobromous acid comprises an oxidizing agent and a solid bromide-releasing compound.

8. A solid composition according to claim 7, wherein the solid bromide-releasing compound comprises at least one selected from the group consisting of brominated hydantoins, bromochlorohydantion—BCDMH, dibromodimethylhydantoin, brominated isocyanurates and bromochloroisocyanuric acid.

9. A solid composition according to claim 3, wherein the bromide salt comprises a bromine-rich sea salt or brine.

10. A solid composition according to claim 7, wherein the bromide-releasing compound comprises at least one selected from the group consisting of ammonium bromide, alkylammonium bromide, dialkylammonium bromide, trialkylammonium bromide, wherein said alkyl radicals are independently selected from straight chain or branched aliphatic, aromatic, or aryl hydrocarbon radicals of between 1 to about 24 carbon atoms.

11. A solid composition according to claim 7, wherein the bromide-releasing compound comprises a bromide ion exchange material.

12. A solid composition according to claim 1, further comprising a solid inorganic source of hypochlorous acid wherein one of the sources is a salt.

13. A solid composition according to claim 1, further comprising a source of hypoiodous acid.

14. A solid composition according to claim 1, further comprising at least one hypochlorite-generator selected from the group consisting of chlorinated trisodium phosphate, chlorinated trisodium polyphosphate, and chlorinated trisodium phosphate dodecahydrate, and mixtures thereof.

15. A solid composition according to claim 1, comprising at least one halohydantoin.

16. A solid composition according to claim 1, comprising at least one halohydantoins selected from the group consisting of 1-bromo-3-chloro-5-methyl-5-ethyl-hydantoin (BCEMH), 1,3-dibromo-5,5-dimethylhydantoin (DBDMH), 1,3-dibromo-5-methyl-5-ethyl-hydantoin (DBEMH), and mixtures thereof.

17. A solid composition according to claim 1, comprising a bromide salt combined with a chloride or iodide salt.

18. A solid composition according to claim 17, wherein the chloride or iodide salt comprises sodium, potassium, lithium, magnesium, calcium, zinc, ammonium, aluminum chloride, and mixtures thereof.

19. A solid composition according to claim 3, comprising about 20 to about 60% by weight sodium chlorite, about 10 to about 60% by weight potassium monopersulfate, and about 10 to about 40% by weight of at least one of sodium bromide or ammonium bromide.

20. A solid composition according to claim 3, wherein the bromide salt is other than calcium bromide and zinc bromide.

21. A solid composition according to claim 1, wherein the solid inorganic source of hypobromous acid and solid source of chlorite are present in an amount such that the pH of a solution formed from dissolving the composition in water is from 6 to 8, measured at 25° C. at a concentration of 1 g of active ingredients of the composition per 100 ml of water.

22. A solid composition according to claim 1, wherein the solid inorganic source of hypobromous acid and solid source of chlorite are present in an amount such that the pH of a solution formed from dissolving the composition in water is from 6.5 to 7.5, measured at 25° C. at a concentration of 1 g of active ingredients of the composition per 100 ml of water.

23. A solid composition according to claim 1, wherein the solid source of chlorite comprises sodium chlorite and the solid inorganic source of hypobromous acid comprises a bromocyanurate.

24. A solid composition according to claim 1, wherein the composition comprises from about 10 to about 90% of the solid inorganic source of hypobromous acid and about 10 to about 90% of the solid source of chlorite.

25. A method of producing chlorine dioxide comprising: adding a composition comprising a solid inorganic source of hypobromous acid, a solid source of chlorite and a free bromine stabilizer, wherein the solid inorganic source of hypobromous acid and solid source of chlorite are present in an amount such that the pH of a solution formed from dissolving the composition in water is from 5 to 9, measured at 25° C. at a concentration of 1 g of active ingredients of the composition per 100 ml of water, the active ingredients being the total weight of the solid inorganic source of hypobromous acid and the solid source of chlorite, to water such that hypobromous acid is produced from the solid inorganic source of hypbromous acid and the hypobromous acid is reacted with chlorite produced from the solid source of chlorite to form chlorine dioxide.

26. A method according to claim 47, further comprising the steps of adding sodium chlorite and potassium monopersulfate to seawater.

27. A method for producing a stabilized solution comprising chlorine dioxide and at least free bromine, the method comprising:
adding to an aqueous solution an oxidizing agent, a source of hypobromous acid, a source of chlorite and a source of free bromine stabilizer, wherein the solid inorganic source of hypobromous acid and solid source of chlorite are present in an amount such that the pH of a solution formed from dissolving the composition in water is from 5 to 9, measured at 25° C. at a concentration of 1 g of active ingredients of the composition per 100 ml of water, the active ingredients being the total weight of the solid inorganic source of hypobromous acid and the solid source of chlorite.

28. A multi-part system for producing chlorine dioxide on demand comprising:
an aqueous solution containing sodium chlorite, sodium bromide, and a free bromine stabilizer; and
a solid comprising at least potassium monopersulfate.

29. A method for producing chlorine-dioxide comprising:
combining an aqueous solution containing sodium chlorite, sodium bromide, and a free bromine stabilizer with a solid comprising at least potassium monopersulfate to thereby produce chlorine dioxide.
